(19) United States
(12) Patent Application Publication
(10) Pub. No.: US 2013/0280376 A1
(43) Pub. Date: Oct. 24, 2013

(54) ENZYME PREPARATION FROM KOJI FERMENTATION

(71) Applicants: Helge Ulmer, Singapore (SG); Beatrice Diono, Singapore (SG); Swen Rabe, Muehlingen (DE)

(72) Inventors: Helge Ulmer, Singapore (SG); Beatrice Diono, Singapore (SG); Swen Rabe, Muehlingen (DE)

(73) Assignee: NESTEC S.A., Vevey (CH)

(21) Appl. No.: 13/997,578

(22) PCT Filed: Dec. 15, 2011
(86) PCT No.: PCT/EP2011/072865

§ 371 Date: Jun. 24, 2013

(30) Foreign Application Priority Data
Dec. 28, 2010 (CN) 201010624763.5

(51) Int. Cl. C12N 9/14; A23L 1/105

(52) U.S. Cl. CPC C12N 9/14; A23L 1/105
USPC 426/18; 435/195; 426/7

(57) ABSTRACT

The invention provides an enzyme preparation obtainable from a Koji fermentation, wherein the Koji fermentation comprises mushrooms fermented with Aspergillus. The invention further relates to an enzyme preparation obtainable from the fermentation of a mixture of mushrooms and cereal with Aspergillus, to a process of producing the enzyme preparation, and to the use of the preparation.

### TECHNICAL FIELD

This invention relates to an enzyme preparation obtainable from a Koji fermentation, which comprises mushrooms fermented with Aspergillus. The invention further relates to an enzyme preparation obtainable from the fermentation of a mixture of mushrooms and cereal, to a process of producing the enzyme preparation and to a use of the preparation.

### BACKGROUND

The hydrolysis of plant proteins represents one of the oldest processes of food biotechnology. For centuries in Asian countries, soy protein has been hydrolysed to produce among other sauces Shoyu, a soy sauce and common condiment which in the Western world has become almost synonymous with Chinese cooking. In these tedious, sometimes perennial processes, the required peptidolytic activities are derived from a consecutive fermentation of Aspergilli, lactic acid bacteria and yeasts growing in the respective plant substrates.

Western countries have imitated this process using wheat, rice or peanut proteins and replacing the enzymatic hydrolysis by acid hydrolysis, for example using hydrochloric acid, to efficiently open the kinetically very stable peptide bonds. The products, usually termed hydrolysed vegetable protein (HVP), are either liquid condiments or concentrated to obtain pastes, powders, pre-mixes for use in soup or sauce compositions, or stock cubes. All of the products impart a meat-like smell and taste, although no meat is involved at any stage of the process.

As the acid has to be neutralised after the hydrolytic process has been completed, sodium hydroxide is added. The sodium chloride formed during this neutralisation reaction supports the taste enhancing properties of the plant hydrolysate and is often welcome in the final product.

However, acid hydrolysis suffers from many disadvantages. Strong acids and alkali are dangerous substances and are regarded as being no longer compatible with safe and food grade operations, especially at temperatures above 100° C. Even more problematic is the inevitable formation of dichloro compounds, such as 1,2- and 1,3-dichloropropanol, which were shown to be carcinogenic in animal feeding studies. The contact of the lipidic fraction of the raw materials with hydrochloric acid also generates chlorinated steroids, while compounds such as 5-(chloromethyl)furfural are derived from Maillard pathways.

In order to avoid the formation of dangerous or toxic compounds, attention has turned again to enzymatic processes where these compounds are neither required nor formed. As a consequence, there has been an immense interest in improving enzymatic processes. Enzymes used in such processes are usually peptidases and/or proteases.

Common peptidases are, for example, alcalase (a serine-peptidase from Bacillus licheniformis), papain (a cystein-peptidase from Carica papaya), chymosin (rennet) from recombinant Mucor strains and neutrase (a metal-peptidase from Bacillus subtilis). U.S. Pat. No. 3,694,316 describes a process for preparing proteases from Basidiomycetes. Other commercial peptidases, such as pronase or Flavorzyme®, are mixtures of various types of activities produced by bacteria, such as Streptomyces griseus, or by ascomycete fungi, such as Aspergillus oryzae.

Due to its biocatalytic nature, the enzymatic process can be performed at gentle physical conditions and is thus more environmentally friendly than its acid catalysed counterpart. However, in contrast to acid hydrolysis, the enzymatic process often does not reach completion and may result in partially hydrolysed products with a bitter taste. The level of bitterness increases with increasing degree of hydrolysis to reach a maximum and then falls with progressing hydrolysis.

Numerous attempts to accelerate the enzymatic process have been published (Pedroche, J. et al., Electronic Journal of Environmental, Agricultural and Food chemistry, 2003) and to avoid the formation of bitter peptides (Maehashi K. et al., Molecular Methods of plant Analysis, 2002, 47-68).

Proteins of plants (Lamsal, B. P. et al., Journal of the American Oil Chemists' Society, 83(8), 731-737, 2006) or animal origin (Je, J. et al., European Food Research and Technology, 221(1-2), 157-162, 2005), or processing wastes such as rice bran have served as substrates (Jarunrattanasri, A. et al., ACS Symposium Series, 83-97, 2005). Substrates such as hemoglobin, casein and insulin have also been investigated (Suzuki, N. et al. Phytochemistry, 2005, 66, 983-990).

Soy and wheat are still the most common enzymatic hydrolysis substrates. If the biocatalyst was not an intact cell, such as for example a micro-organism, enzyme preparations, such as neutrase (WO 2006/103628), or combinations of plant and microbial enzymes (WO 2002/078461; CN 1397644) were suggested.

Peptidolysis of wheat gluten in particular has been achieved using alcalase, pancreatin or pepsin (Kong, X. et al., Food Chemistry, 101, 615-620, 2007), or papain (Wang, J. et al., Food Chemistry, 101, 1658-1663, 2007). Finally, it is also known to hydrolyse wheat gluten enzymatically using commercially available enzyme preparations, for example Flavorzyme®. The critical points in the development of WGH-te (Wheat Gluten Hydrolysate—technical enzymes) processes, however, are still mainly the high costs of these enzymes, the insufficient proteolytic activity of available enzyme preparations in comparison to, for example, the acid hydrolysis processes, the insufficient digestion of insoluble residues (for example, fractions rich in glycoproteins) in the hydrolysate, and the microbiological protection of the hydrolysates since they are run without or at low salt concentrations, and at temperatures which are permissible for certain spoilage micro-organisms to grow.

Consequently, there has been a need for an enzyme preparation with improved properties for the hydrolysis of cereal products.

The applicant has surprisingly found that an enzyme preparation obtainable from a Koji fermentation, based on mushrooms as a raw material for the fermentation process, increased the expression of proteases and/or peptidases from Aspergillus. The presence of a cereal product, for example wheat or barley, in the fermentation process further increased the proteolytic activity of these enzymes. Still further production and/or activity increases could be obtained by adding a small amount of a growth medium to the fermentation process.

The crude enzyme preparation obtained in this way was more active than commercial enzyme preparations, for example Flavorzyme®, and lead to an enhanced and faster proteolytic activity against wheat gluten. This allows the use of this enzyme preparation in a more efficient hydrolysis process of cereal products, such as wheat or soy, resulting in improved hydrolysis of those raw materials. The hydrolysis process was faster, thereby reducing the risk of spoilage by undesired micro-organisms and reducing the cost of production by making use of a crude enzyme preparation instead of using a purified commercial enzyme. Furthermore, and contrary to the use of potent commercial enzymes, for example Alcalses, no off-flavours, i.e. bitterness, were detected.

An object of the present invention is therefore to provide an enzyme preparation that at least goes part way to overcoming one or more of the above disadvantages of known enzyme preparations.

### SUMMARY OF THE INVENTION

In a first aspect, the invention provides an enzyme preparation obtainable from a Koji fermentation, wherein the Koji fermentation comprises mushrooms fermented with Aspergillus.

In a second aspect, the invention provides a process for the production of an enzyme preparation comprising the step of fermenting mushrooms with Aspergillus, preferably fermenting mushrooms together with cereal.

A further aspect of the invention provides the use of the enzyme preparation of the invention for the hydrolysis of cereal products or meat products, and for the preparation of food products.

### BRIEF DESCRIPTION OF THE DRAWINGS

FIG. 1: Process steps for preparing the crude enzyme preparation of the invention.

FIG. 2: Protein fractionation of the crude enzyme preparation after elution through a Sepharose Fast Flow chromatography system.

FIG. 3: Proteolytic activity of enzymes from mushroom-based koji preparation compared to a Flavourzyme™ preparation. Values are normalised to 100% of the Flavourzyme™ activity.

### DETAILED DESCRIPTION

The invention relates to an enzyme preparation obtainable from a Koji fermentation, where the Koji fermentation comprises fermenting mushrooms with Aspergillus. Preferably, the Koji fermentation further comprises a cereal fermented with Aspergillus.

The term “Koji fermentation” refers to a fermentation process, for example a solid state fermentation process, of soybean, wheat, barley and/or rice with a mould such as an Apergillus for the production of various traditional food and beverage products, for example miso, soy sauce, sake, and the like.

The Koji fermentation of the invention comprises mushrooms or a mixture of mushrooms preferably with at least one cereal selected from the group consisting of wheat, barley, rice or a combination thereof. The inventors surprisingly found that the presence of mushrooms in the fermentation mixture generated a Koji with a significantly enhanced proteolytic activity against gluten from cereals. The mixture may comprise from about 10 to 99 wt % mushrooms, preferably from about 60-90 wt % mushrooms, and/or from about 10 to 60 wt % cereal, preferably from about 10-40 wt % cereal. In one embodiment, the mixture comprises about 90 wt % mushrooms and 10 wt % of a cereal, where wheat is the preferred cereal. The addition of a cereal product was found to be advantageous for the consistency and air permeability of, for example, solid state Koji fermentation. This allows a better growth of the Aspergillus mould in the Koji, a better hydration of the Koji bed and control of the fermentation temperature, for example.

The cereal may be selected from the group consisting of whole cereal, cereal gluten, cereal bran, cereal husks, cereal rootlets, processed cereal, or any combination thereof.

The activity of the enzyme preparation can be further increased by the addition of low amounts of a growth medium to the Koji fermentation. The advantage is that such growth media further enhance the obtainable enzyme yield and activity of the fermentation process as well as the stability of the Koji fermentation process. Hence, the Koji fermentation of the invention further comprises such a growth medium in a range from about 0.1 to 10 wt %, preferably from about 1 to 6 wt %, and even more preferably from 1 to about 3 wt % of the total mixture. The growth medium is selected from the group consisting of defatted soybean cake, yeast extract, peptone, corn steep liquor, or any combination thereof.

The Koji fermentation of the invention may comprise a mushroom or a mixture of mushrooms, whereby the mushrooms may be preferably selected from the group consisting of Coprinus comatus, Lentinula edodes, Agaricus bisporus, Agaricus campestris, Hypsizygus tessulatus, Pleurotus ostreatus, Pleurotus citrinipileatus, Pleurotus eryngii, Boletus edulis, Craterellus cornucopioides, Craterellus tubaeformis, Agaricus blazei, Volvariella volvacea, Agrocybe Aegerita and Ganoderma lucidum, or any combination thereof. The mushrooms or the mixture of the mushrooms can comprise fresh mushrooms, dried or processed mushrooms, whole mushrooms or any parts of the mushrooms such as only stems or fruit bodies, and/or any combination of the above. The advantages of using dried or processed mushrooms versus fresh mushrooms are better storage and easier processing of this plant material in an industrial setting. Further advantages may be price and the availability of such raw materials all around the year.

The Koji of the invention is preferably fermented with the filamentous fungus Aspergillus oryzae. Aspergillus oryzae has the advantage of being a well recognised filamentous fungus used in traditional Koji fermentations. It is absolutely safe for food consumption and provides typical fermented soy sauce type flavour notes.

A further aspect of the invention is a process for the production of an enzyme preparation described in the present invention comprising the step of fermenting a mixture of mushrooms and at least one cereal with an Aspergillus. Thus, a mixture of mushrooms with at least one cereal can be inoculated with an Aspergillus prior to the fermentation process. Alternatively, mushrooms inoculated with an Aspergillus can be mixed with a cereal prior to the fermentation process. The crude enzyme preparation obtained by this process can be applied directly to the use as described below. The advantage of this process is that a crude enzyme preparation can be obtained which is more active than any generally known commercial enzyme preparations currently used in industrial wheat gluten hydrolysis processes. A further advantage is that no off-flavour, such as bitterness, was detected in the hydrolysis products of the invention.

The process may further comprise the step of at least partially purifying the enzyme preparation by, for example, extraction, separation or concentration, or any combination of these technologies. The enzyme preparation may be, for example, separated and concentrated by DEAE-Sepharose chromatography. The separation and condensation steps of the enzyme preparation may also comprise membrane separation technology. The advantage is that the activity of the enzyme preparations can, for example, be further increased, which would allow even better hydrolysate yields on selected raw materials. Further, any storage, transportation from, for example, one factory location to another factory location and/or handling at a factory site may be facilitated and cheaper.

A still further aspect of the invention is the use of the enzyme preparation of the invention for the hydrolysis of cereal products, such as wheat or soy products for example. The use of the enzyme preparation enables improvements to the hydrolysation process of cereal gluten, including, for example, yield, speed and cost of the fermentation process, to reduce the risk of spoilage by undesired micro-organisms and to improve the taste and reduce any off-taste of the hydrolysed products.

Hence, more specifically, the use of the enzyme preparation relates to the preparation of a food product such as a seasoning sauce, taste maker, soup, dehydrated seasoning, bouillon or paste. The crude enzyme preparation obtainable by the process of the invention can be applied.

Those skilled in the art will understand that they can freely combine all features of the present invention disclosed herein. In particular, features described for the product of the invention may be combined with the method of the invention and vice versa. Further, features described for different embodiments of the present invention may be combined.

Further advantages and features of the invention are apparent from the figures and examples.

### EXAMPLES

The invention is further described with reference to the following examples. It will be appreciated that the invention as claimed is not intended to be limited in any way by these examples.

### Example 1

### Crude Enzyme Preparation

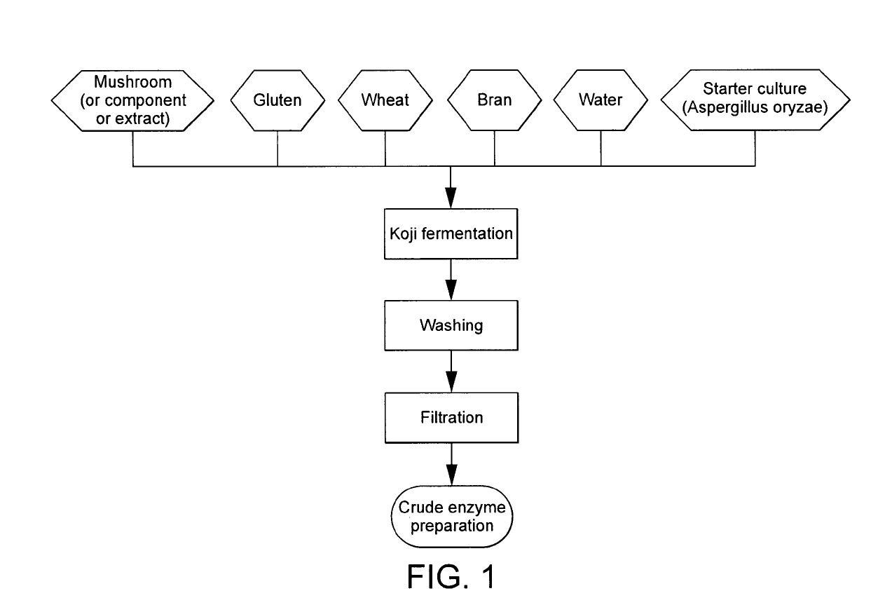

Crude enzyme preparations were prepared according to FIG. 1. Samples of Koji substrates were first prepared by mixing a mushroom base (Shaggy Mane mushroom (Coprinus comatus)), wheat gluten, whole wheat and wheat bran in the ratios as indicated in Table 1. Then, 10 g of each resulting Koji substrate was mixed with 5 g water, inoculated with spores of Aspergillus oryzae and thereafter fermented in a traditional solid-state Koji fermentation system. The fermentation was carried out at 30° C. for 30 h at a water humidity of 80% RH (relative humidity). Then, the fermented Koji was washed with 80 ml water at 4° C. for 1 h. The Koji was then filtered and the retentate retained as the crude enzyme preparation.

TABLE 1
Koji compositions

| | Wheat bran | Mushroom base | Wheat gluten | Whole wheat |
|---|---|---|---|---|
| Sample | (%) | (%) | (%) | (%) |
| 1 | 10 | 90 | 0 | 0 |
| 2 | 10 | 67.5 | 22.5 | 0 |
| 3 | 10 | 67.5 | 0 | 22.5 |
| 4 | 10 | 45 | 45 | 0 |
| 5 | 10 | 45 | 22.5 | 22.5 |
| 6 | 10 | 45 | 0 | 45 |
| 7 | 10 | 22.5 | 45 | 22.5 |
| 8 | 10 | 22.5 | 22.5 | 45 |
| 9 | 10 | 0 | 45 | 45 |

### Example 2

### Enzyme Assay

The enzymatic activity of the different crude enzyme preparations was determined as follows. 20 ml of crude enzyme preparation was added to 1 g of wheat gluten in 30 ml of 0.2 M phosphate buffer (at pH 7.2), and the subsequent mixture was inoculated at 50° C. for 30 min. Thereafter, the mix was heated for 10 min at 100° C. in order to terminate any further enzymatic reactions. Formol nitrogen (FN) was then determined according to the protocol provided by the reference Hawk, P. B., Oser, B. L., and Summerson, W. H. Henriques-Sørensen, Formol titration method: principle, In Practical Physiological Chemistry, Thirteenth edition, New York, Blakiston, 1954. pp. 897-899. The formol value gives an estimate of the amount of free aminoacids generated in a solution. As a blank sample for the enzymatic measurements, the crude enzyme preparations were first heated for 10 min to 100° C. in order to inactive the enzymes, and thereafter processed in the same way as the samples used to measure the formol nitrogen content. The enzyme activity values were calculated as follows:

ΔFN=[(FN of sample)−(FN of blank)]/wheat gluten[mg/g] and per minute incubation.

The results are given in Table 2.

TABLE 2
Impact of crude enzyme preparations on wheat gluten hydrolysis

| | ΔFN |
|---|---|
| Sample | [μg/g * min] |
| 1 | 65 |
| 2 | 52 |
| 3 | 60 |
| 4 | 38 |
| 5 | 40 |
| 6 | 37 |
| 7 | 26 |
| 8 | 14 |
| 9 | 11 |

Formol Nitrogen (FN) released during enzymatic hydrolysis was monitored as indicator of proteolysis. It was found that the most important ingredient in the fermented Koji base for the optimisation of proteolysis of the wheat gluten solution was the mushroom base. The best result was obtained with the Koji sample 1 product. Consistent with this result, it was also observed that the wheat gluten solutions after the hydrolysis step were clearest, i.e. most transparent samples, when hydrolysed with the enzyme mix from the Koji mixture with the highest mushroom contents. It can be concluded that whenever a mushroom base is present in the fermented Koji, subsequent hydrolysis of a wheat gluten solution is improved. Best results are achieved with a high percentage of such a mushroom base, as for example in sample 1 with 90 wt % mushrooms and 10 wt % wheat bran, in the fermented Koji.

### Example 3

A Koji substrate was prepared with mushrooms as main component (about 90 wt %), wheat bran (10 wt %) (sample 1 in Example 1), and various supplements present in different amounts. The enzyme activity for hydrolysis of wheat gluten from different fermentation experiments was determined by orthogonal experimental design, and some of the results are presented in Table 3. The values present the average difference obtained from the control sample, i.e. the fermentation without a supplement (sample 1 from Example 2), and those with a supplement of 2 wt % of defatted soybean, yeast extract or peptone.

TABLE 3
Impact on formol nitrogen (FN) production from wheat gluten
of samples supplemented with defatted soybean, yeast extract
or peptone.

| | Defatted soybean | | |
|---|---|---|---|
| | cake | Yeast extract | Peptone |
| Difference in ΔFN | +5.9 [μg/g * min] | +5.57 | +13.3 |
| from control | | [μg/g * min] | [μg/g * min] |
| sample | | | |

Among the tested supplements, peptone had the strongest positive effect on the production of FN. Peptone promoted the expression of exo-proteases, with a strong positive effect on the release of free amino acids of the tested wheat gluten solution. Defatted soybean cake and yeast extract promoted the expression of exo-proteases as well, despite the fact that the overall impact on the proteolytic activity was not as strong as that observed for the peptone. Hence, the addition of certain selected supplements to the Koji fermentation can further improve the proteolytic effect of the enzyme preparation.

### Example 4

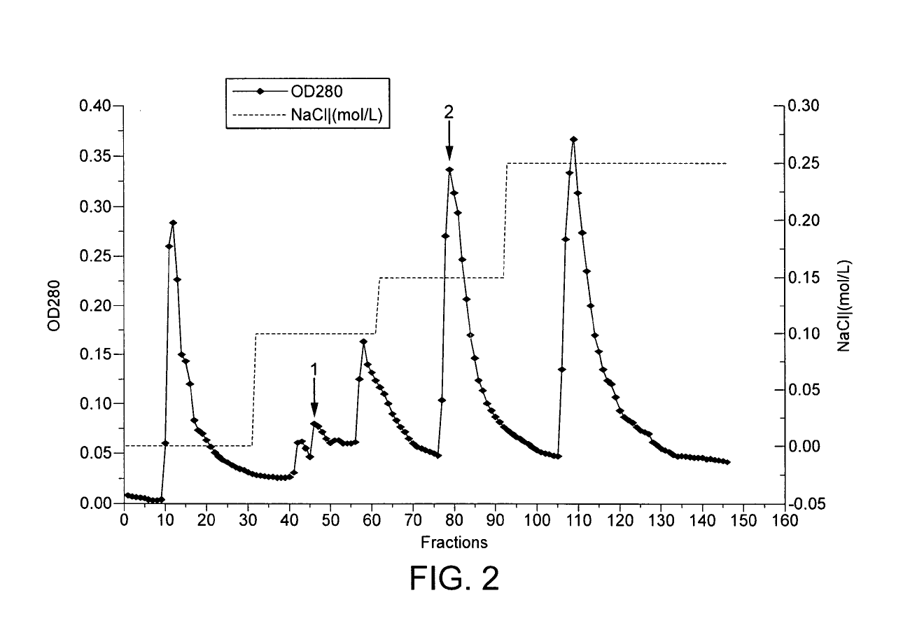

Crude enzyme preparation of Example 1 was run on a DEAE-Sepharose Fast Flow chromatography system. FIG. 2 shows the result. Each of the peaks in the graph represents proteins that were present in the enzyme preparation. Proteolytic activity was assayed for each of the protein peaks, and only the one peak as indicated in FIG. 2 by an arrow “2” was found to have significant protease activity. Biochemical purification of the protease from the fermented mushroom-based Koji was achieved by ammonium sulfate precipitation and subsequent DEAE-Sephadex A50 and Butyl-Sepharose CL-4B chromatography according to standard procedures known by the skilled person.

Characteristics of the protease isolated from the mushroom-based Koji were:
Relative molecular weight: 53.8 kDa (based on SDS-PAGE and ion-trap MS LCQ Deca XP)
Isoelectric point: 4.1-4.2
Specific activity: 2274.7 U/mg

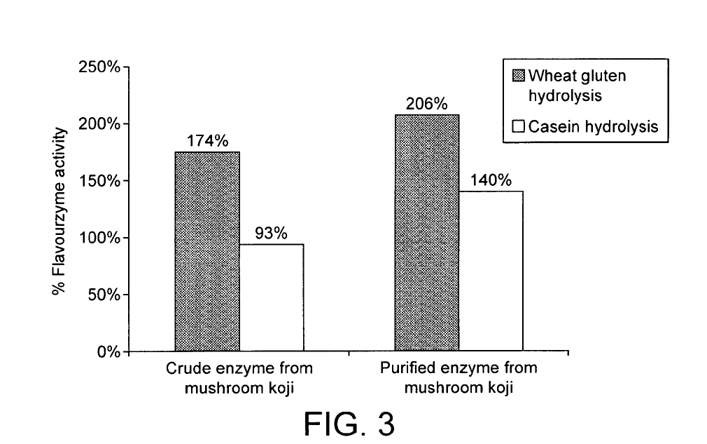

This partially purified preparation resulted in an enzyme preparation with an increased enzyme activity of 67.1 times. The recovery yield of the enzyme in the purification process was 11.3%. The proteolytic activity of the subsequent partially purified enzyme preparation produced by a mushroom-based Koji was compared to the proteolytic activity of the commercially available purified Flavourzyme™, when hydrolysing either wheat gluten or casein. The result is shown in FIG. 3, wherein the proteolytic activity of Flavourzyme™ is taken as 100% and compared to the partially purified enzyme preparation of the invention. The proteolytic activity of the partially purified enzyme preparation produced by a mushroom-based Koji was therefore superior to the commercial and broadly used Flavourzyme™.

### Example 5

Koji substrate (containing dried mushroom and wheat gluten) was mixed to water, inoculated with spores of Aspergillus oryzae, and incubated at 30° C. and 80% RH for 30 h. Koji was harvested, mixed to water and wheat gluten, and held at 50° C. for 3 h. The solution was filtered and the reaction stopped by heating the supernatant at over 90° C. for 10 min. The produced sauce was evaluated by informal tasting. Typical flavour notes of hydrolyzed wheat gluten sauce were achieved and no bitter off-flavours were perceived.

It is to be appreciated that although the invention has been described with reference to specific embodiments, variations and modifications may be made without departing from the scope of the invention as defined in the claims. Furthermore, where known equivalents exist to specific features, such equivalents are incorporated as if specifically referred to in this specification.

1. An enzyme preparation obtained from a Koji fermentation, wherein the Koji fermentation comprises mushrooms fermented with Aspergillus.

2. The enzyme preparation according to claim 1, wherein the Koji fermentation comprises a cereal fermented with Aspergillus.

3. The enzyme preparation according to claim 2, wherein the cereal is selected from the group consisting of wheat, barley, and combinations thereof.

4. The enzyme preparation according to claim 1, wherein the Koji fermentation comprises 10-99 wt % mushrooms and 10-60 wt % cereal.

5. The enzyme preparation according to claim 1, wherein the Koji fermentation comprises 60-90 wt % mushrooms and 10-40 wt % cereal.

6. The enzyme preparation according to claim 1, wherein the Koji fermentation comprises 90 wt % mushrooms and 10 wt % cereal.

7. The enzyme preparation according to claim 2, wherein the cereal is wheat.

8. The enzyme preparation according to claim 1, wherein the Koji fermentation comprises a growth medium comprising from 0.1-10 wt % of the total mixture.

9. The enzyme preparation according to claim 8, wherein the growth medium comprises from 1-3 wt % of the total mixture.

10. The enzyme preparation according to claim 8, wherein the growth medium is selected from the group consisting of defatted soybean cake, yeast extract, peptone, corn steep liquor, and combinations thereof.

11. The enzyme preparation according to claim 1, wherein the mushroom is selected from the group consisting of Agaricus brunnescens, Coprinus comatus, Lentinula edodes, Agaricus bisporus, Agaricus campestris, Hypsizygus tessulatus, Pleurotus ostreatus, Pleurotus citrinipileatus, Pleurotus eryngii, Boletus edulis, Craterellus cornucopioides, Craterellus tubaeformis, Agaricus blazei, Volvariella volvacea, Agrocybe Aegerita or Ganoderma lucidum, and combinations thereof.

12. The enzyme preparation according to claim 2, wherein the cereal is selected from the group consisting of whole cereal, cereal gluten, cereal bran, cereal husks and rootlets, and processed cereal, and combinations thereof.

13. The enzyme preparation according to claim 1, wherein the Aspergillus is Aspergillus oryzae.

14. A process for producing an enzyme preparation comprising the step of fermenting a mushroom with Aspergillus to produce a Koji fermentation.

15. The process according to claim 14, wherein a cereal is included in the fermenting step.

16. The process according to claim 14, comprising the step of purifying the enzyme preparation by a step selected from the group consisting of extraction, separation, concentration, and combinations thereof.

17. A method for the hydrolysis of a cereal product, a meat product, or a combination thereof comprising using an enzyme preparation obtained from a Koji fermentation, wherein the Koji fermentation comprises mushrooms fermented with Aspergillus.

18. A method for the preparation of a food product such as a seasoning sauce, taste maker, soup, dehydrated seasoning, bouillon or paste comprising using an enzyme preparation obtained from a Koji fermentation, wherein the Koji fermentation comprises mushrooms fermented with Aspergillus.
